(19) United States
(12) Patent Application Publication
(10) Pub. No.: US 2026/0337540 A1
(43) Pub. Date: Oct. 29, 2026

(54) FOOD WASTE DISPOSER WITH BY-PRODUCT EMPTYING NOTIFICATION

(71) Applicant: Hanmi Flexible Co., Ltd., Gimhae-si (KR)

(72) Inventors: Heon Pyeong JI, Busan (KR); Da Hun KIM, Gimhae-si (KR); Sang Hyun KIM, Gimhae-si (KR)

(73) Assignee: Hanmi Flexible Co., Ltd., Gimhae-si (KR)

(21) Appl. No.: 19/261,884

(22) Filed: Jul. 7, 2025

(30) Foreign Application Priority Data
Apr. 23, 2025 (KR) 10-2025-0052926

(51) Int. Cl. C12M 1/06; B01F 27/90; B01F 35/21; B01F 35/22; B09B 3/60; B09B 101/70; B65F 1/02; B65F 1/14; C12M 1/34

(52) U.S. Cl. CPC C12M 27/06; B01F 27/90; B01F 35/2112; B01F 35/2209; B09B 3/60; B65F 1/02; B65F 1/14; C12M 41/44; B09B 2101/70; B65F 2210/168

(57) ABSTRACT

Embodiments of the present disclosure relate to a food waste disposer with by-product emptying notification, and more specifically, to a food waste disposer with by-product emptying notification which detects by-products stored while food waste is fermented and decomposed inside a storage tank on an outer side wall of the storage tank, and displays the height of the by-products or the state of the food waste disposer, thereby improving the durability and accuracy of a detection sensor.

### CROSS-REFERENCE TO RELATED PATENT APPLICATION

The present application claims priority under 35 U.S.C. § 119 (a) to Korean patent application number 10-2025-0052926 filed on Apr. 23, 2025, in the Korean Intellectual Property Office, the entire disclosure of which is incorporated by reference herein.

### BACKGROUND OF THE INVENTION

### 1. Field

Embodiments of the present disclosure relate to a food waste disposer, and more specifically, to a food waste disposer which detects by-products accumulated as food waste is fermented and decomposed inside a storage tank on an outer side wall of the storage tank and displays the height of the by-products or the state of the food waste disposer, thereby notifying when the by-products should be emptied.

### 2. Description of the Related Art

In general, food waste generated after meals and cooking at home or in restaurants is managed separately from general waste due to environmental pollution and sanitary issues. In other words, food waste is required to be collected separately from general waste or to be hygienically managed or disposed of by applying different costs depending on the amount of food waste discharged.

In addition, since the food waste collection location is usually a specific outdoor location in apartment complexes, the inconvenience of frequent movement often leads to the storage of food waste in households until a certain amount is reached and then processed at once, which has a negative impact on the indoor environment.

For these management and economic reasons, methods for processing food waste indoors on their own have been developed recently, and as one of them, research is actively being conducted on an eco-friendly food waste disposer that temporarily stores food waste in a storage tank where an atmosphere is created that activates microorganisms, naturally decomposes food waste by microorganisms, and then processes the by-products.

In the case of an eco-friendly food waste disposer, the time comes to empty the storage tank when it is full of by-products, and in order to solve the problem that the user has to visually check whether the by-products have reached the appropriate level and empty them, food waste disposers that have introduced a level sensor or an overload detection sensor, such as Korean Patent Application Publication No. 10-2020-0040573 and Korean Patent Application Publication No. 10-2024-0080217, have been filed, but such food waste disposers detect by-products by operating a sensor located inside the storage tank to detect the by-products, and the inside of the storage tank is vulnerable to dust and waterproofing, so they have drawbacks in that the durability of the sensor is low and the accuracy of sensing is low due to temperature, dust scattering, irregular reflection, the sedimentation of the by-products, and the like.

Accordingly, the need for a food waste disposer that ferments and decomposes food waste inside the storage tank, improves durability, and more accurately measures the stored by-products to display the height of the by-products or the state of the food waste disposer to notify when the by-products should be emptied is more important.

### PRIOR-ART DOCUMENTS

### Patent Documents

(Patent Document 1) Korean Patent Application Publication No. 10-2020-0040573A
(Patent Document 2) Korean Patent Application Publication No. 10-2024-0080217A
(Patent Document 3) Korean Patent No. 10-2762398B1

### SUMMARY OF THE INVENTION

The present disclosure has been devised to solve the problems described above, and more specifically, to provide a food waste disposer with by-product emptying notification that detects by-products accumulated as food waste is fermented and decomposed inside a storage tank on an outer side wall of the storage tank, and displays the height of the by-products or the state of the food waste disposer, thereby improving the durability and accuracy of a detection sensor.

A food waste disposer with by-product emptying notification according to an embodiment of the present disclosure includes:
a housing 100 having an upper part that opens and closes and a space formed inside; a storage tank 200 coupled inside the housing 100 and configured to store by-products A of food waste; a stirring rod 300 rotatably driven inside the storage tank 200; at least one stirring blade 400 protrudingly formed on an outer circumferential surface of the stirring rod 300 and configured to stir the by-products A stored in the storage tank 200 by rotation of the stirring rod 300; a sensor 500 configured to detect the by-products A inside the storage tank 200; a controller configured to control an operation of the food waste disposer in conjunction with the sensor 500; and a display 600 configured to receive information from the controller and display a height of the by-products A or a state of the food waste disposer, wherein the sensor 500 is located on an outer side wall of the storage tank 200, and its attachment position is adjustable.

More specifically, the food waste disposer may further include a guide rail 510 formed along an outer circumferential surface of the storage tank 200, wherein the guide rail 510 may be vertically formed in an upward direction of a central axis of the stirring rod 300, and the sensor 500 may be moved or fixed by sliding engagement with the guide rail 510.

More specifically, at a first position where the sensor 500 is set, in the case that the sensor 500 continuously detects the presence of the by-products A at a location inside the storage tank 200 corresponding to the position of the sensor 500 for a predetermined period of time, the controller may determine that the by-products A are present up to a height of the first position.

More specifically, when the controller determines that the by-products A are present up to the height of the first position, the controller may control the upper part of the housing 100 to be in a locked state, and the upper part of the housing 100 may be configured to be opened through unlocking by a user.

More specifically, the sensor 500 may be configured to detect the presence of the by-products A stored at a location inside the storage tank 200 corresponding to a position of the sensor 500.

More specifically, the controller may be provided with information on a deposition height or capacity inside the storage tank 200 according to a position of the sensor 500 in advance, and in the case that the sensor 500 continuously detects the presence of the by-products A inside the storage tank 200 for a predetermined period of time, the controller may obtain the deposition height or capacity of the by-products A inside the storage tank 200 and display it on the display 600.

More specifically, the sensor 500 may be a capacitive sensor.

More specifically, the display 600 may be configured to display a notification of the height of the by-products A inside the storage tank 200 in a visual or auditory manner.

A food waste disposer with by-product emptying notification according to an embodiment of the present disclosure detects by-products accumulated as food waste is fermented and decomposed inside a storage tank by a sensor, and displays the height of the by-products or the state of the food waste disposer, thereby providing the effect of notifying an appropriate time to empty the by-products.

In particular, a food waste disposer with by-product emptying notification according to an embodiment detects by-products stored inside a storage tank by a sensor located on an outer side wall of the storage tank rather than inside the storage tank, thereby providing the effect of improving the durability and accuracy of the detection sensor.

In particular, a food waste disposer with by-product emptying notification according to an embodiment has a sensor located on the central axis line of a stirring rod on the outer side wall of the storage tank to minimize the influence of by-products flying and depositing due to the stirring operation of the stirring blade, thereby enabling more accurate measurement of the deposition height. In other words, since the vicinity of the central axis line of the stirring rod, where the sensor is installed, is the most stable location for by-products to be deposited, it provides the effect of improving the accuracy of deposition height measurement.

In addition, a food waste disposer with by-product emptying notification according to an embodiment has a sensor located on the outer side wall of the storage tank and configured such that its attachment position is adjustable, thereby providing an effect of sensing at various positions depending on the situation even with a single sensor.

### BRIEF DESCRIPTION OF THE DRAWINGS

FIG. 1 is a perspective view illustrating a food waste disposer with by-product emptying notification according to an embodiment of the present disclosure.

FIG. 2 is a side cross-sectional view for explaining a food waste disposer with by-product emptying notification according to an embodiment of the present disclosure.

FIG. 3 is a front cross-sectional view for explaining a food waste disposer with by-product emptying notification according to an embodiment of the present disclosure.

FIG. 4 is a diagram for specifically explaining a sensor and a guide rail according to an embodiment of the present disclosure, FIG. 4A is a diagram for explaining a sensor located on an outer side wall of a storage tank in a lateral direction, FIG. 4B is a diagram for explaining a sensor located on an outer side wall of a storage tank in a frontal direction, and FIG. 4C is a diagram illustrating an embodiment of a sensor slidably engaged with a guide rail.

FIG. 5 is a diagram for specifically explaining an operation of a sensor according to an embodiment of the present disclosure, FIG. 5A is a diagram illustrating a situation in which a sensor is sensing at a first position and a height of by-products stored in a storage tank has not reached the first position, and FIG. 5B is an enlarged schematic diagram of the sensor at the first position of FIG. 5A.

FIG. 6 is a diagram for specifically explaining an operation of a sensor according to an embodiment of the present disclosure, FIG. 6A is a diagram illustrating a situation in which a sensor is sensing at a first position and a height of by-products stored in a storage tank has reached the first position, and FIG. 6B is an enlarged schematic diagram of the sensor at the first position of FIG. 6A.

### DETAILED DESCRIPTION

The following detailed description of the present disclosure refers to the accompanying drawings, which illustrate specific embodiments in which the present disclosure may be practiced. These embodiments are described in sufficient detail to enable those skilled in the art to practice the present disclosure. It should be understood that the various embodiments of the present disclosure, while different from one another, are not necessarily mutually exclusive. For example, the specific shapes, structures, and characteristics described herein may be implemented in other embodiments without departing from the spirit and scope of the present disclosure with respect to an embodiment.

In addition, it should be understood that the location or arrangement of individual components within each disclosed embodiment may be changed without departing from the spirit and scope of the present disclosure. Accordingly, the following detailed description is not intended to be limited, and the scope of the present disclosure is defined only by the appended claims, along with the full range of equivalents to which such claims are entitled, if appropriately described. Like reference numerals in the drawings designate the same or similar functions throughout the several aspects.

Hereinafter, in order to enable those skilled in the art to easily practice the present disclosure, preferred embodiments of the present disclosure will be described in detail with reference to the accompanying drawings.

In the present specification, by-product (A) is explained as a concept that includes all reactants formed by the reaction between food waste and microorganisms.

<Food Waste Disposer with by-Product Emptying Notification According to an Embodiment of the Present Disclosure>

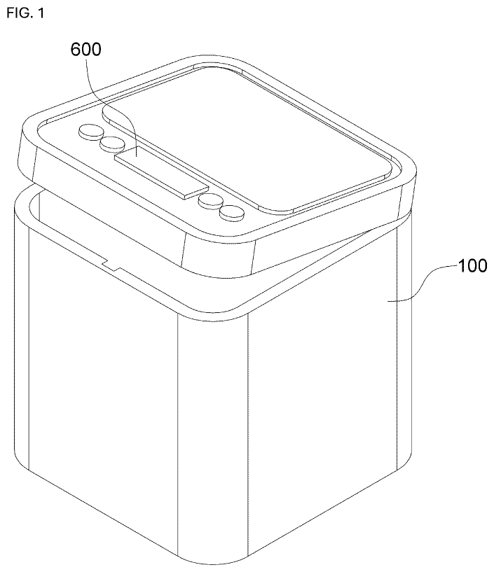

FIG. 1 is a perspective view illustrating a food waste disposer with by-product emptying notification according to an embodiment of the present disclosure.

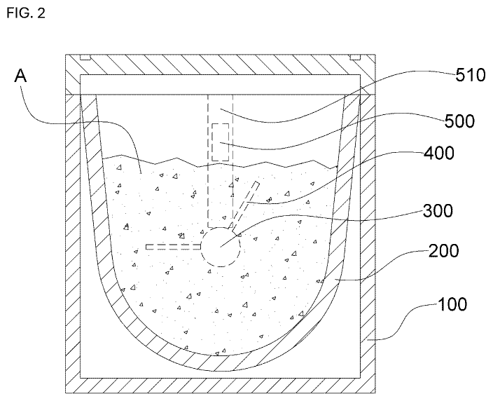

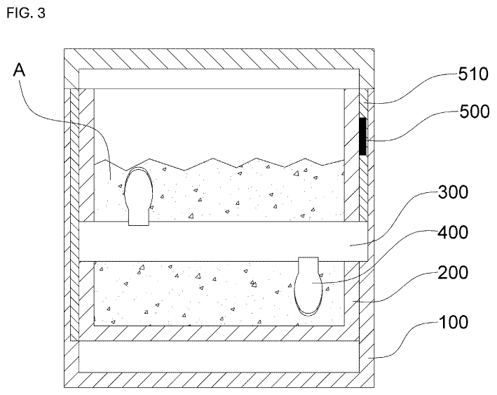

FIG. 2 is a side cross-sectional view illustrating a food waste disposer that indicates the time of emptying by-products according to an embodiment of the present invention, and FIG. 3 is a front cross-sectional view illustrating a food waste disposer that indicates the time of emptying by-products according to an embodiment of the present invention.

Referring to FIGS. 1 to 3, a food waste disposer with by-product emptying notification according to an embodiment of the present disclosure may be configured to include a housing 100, a storage tank 200, a stirring rod 300, a stirring blade 400, a sensor 500, a controller, and a display 600.

The housing 100 is an outer case of the food waste disposer, and as illustrated in FIG. 1, may be configured such that its upper part is opened and closed and a space is formed inside. It is preferable that the housing 100 be manufactured in a hexahedral shape, but it is not necessarily limited thereto.

The upper part of the housing 100 is openable, and it is preferable that part or all of the upper part be opened and closed by a hinge joint, but it is not necessarily limited thereto, and part or all of the upper part may be configured to be opened and closed by sliding.

In addition, the upper part of the housing 100 may be configured to be opened and closed automatically or manually, and may be configured so that opening and closing is restricted by a separate locking device.

The storage tank 200 is a space where food waste input by a user, microorganisms provided to ferment and decompose food waste, and by-products A, which are reactants formed by a reaction between food waste and microorganisms, are stored, and as illustrated in FIGS. 2 and 3, it may be configured to be coupled inside the housing 100 so that the by-products A are stored.

Specifically, it is preferable that the storage tank 200 be formed with an open upper part and a lower part with a semi-cylindrical shape so that by-products (A) may be accommodated, but it is not necessarily limited thereto.

In addition, the storage tank 200 may be formed of a plastic material such as polypropylene (PP), polyethylene (PE), acrylonitrile butadiene styrene (ABS), glass fiber reinforced plastic (PC), or stainless steel in consideration of corrosion resistance, durability, thermal conductivity, and noise.

FIG. 2 is a side cross-sectional view illustrating a food waste disposer that indicates the time of emptying by-products according to an embodiment of the present invention, and FIG. 3 is a front cross-sectional view illustrating a food waste disposer that indicates the time of emptying by-products according to an embodiment of the present invention.

The stirring rod 300 and the stirring blade 400 are for improving the reactivity between food waste and microorganisms by stirring the by-products A stored in the storage tank 200 described above, and referring to FIGS. 2 and 3, the stirring rod 300 may be configured to be rotatably driven inside the storage tank 200.

The stirring rod 300 may be installed vertically between the parallel facing surfaces of the storage tank 200, and specifically, it is preferably installed on a semi-cylindrical shaft of the lower part of the storage tank 200.

The stirring blade 400 is protrudingly formed on an outer circumferential surface of the stirring rod 300, and may be configured to stir the by-products A stored in the storage tank 200 by the rotation of the stirring rod 300.

At least one stirring blade 400 may be provided.

These stirring blades 400 may be disposed to be spaced apart from each other at a certain interval in the longitudinal direction of the stirring rod 300, and it is preferable that the longitudinal direction of the stirring blades 400 protruding from the outer circumferential surface of the stirring rod 300 for at least one of the stirring blades 400 is not the same, but is not necessarily limited thereto.

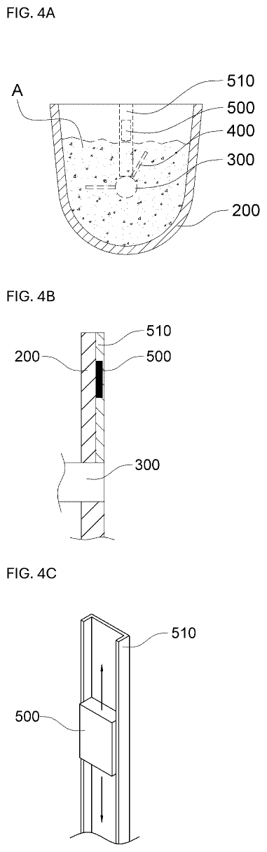

FIG. 4 is a diagram for specifically explaining a sensor and a guide rail according to an embodiment of the present disclosure, FIG. 4A is a diagram for explaining a sensor located on an outer side wall of a storage tank in a lateral direction, FIG. 4B is a diagram for explaining a sensor located on an outer side wall of a storage tank in a frontal direction, and FIG. 4C is a diagram illustrating an embodiment of a sensor slidably engaged with a guide rail.

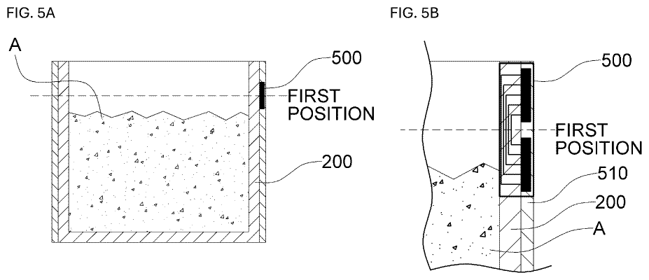

FIG. 5 is a diagram for specifically explaining an operation of a sensor according to an embodiment of the present disclosure, FIG. 5A is a diagram illustrating a situation in which a sensor is sensing at a first position and a height of by-products stored in a storage tank has not reached the first position, and FIG. 5B is an enlarged schematic diagram of the sensor at the first position of FIG. 5A.

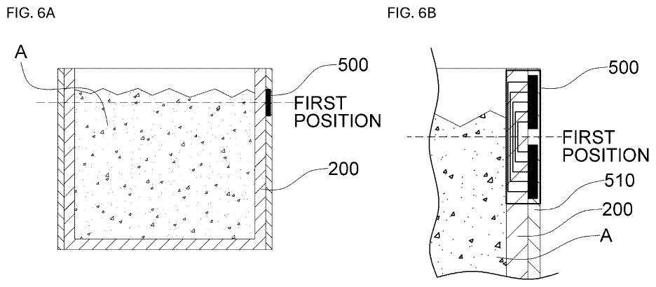

FIG. 6 is a diagram for specifically explaining an operation of a sensor according to an embodiment of the present disclosure, FIG. 6A is a diagram illustrating a situation in which a sensor is sensing at a first position and a height of by-products stored in a storage tank has reached the first position, and FIG. 6B is an enlarged schematic diagram of the sensor at the first position of FIG. 6A.

Referring to FIGS. 4 to 6, the sensor 500 is for detecting by-products A inside the storage tank 200, and the sensor 500 may be located on the outer side wall of the storage tank 200 and configured such that its attachment position is adjustable.

To this end, the food waste disposer with by-product emptying notification according to an embodiment of the present disclosure further includes a guide rail 510 formed along the outer circumferential surface of the storage tank 200, as illustrated in FIG. 4, and the sensor 500 may be configured to be moved or fixed in height by sliding engagement with the guide rail 510.

In addition, the sensor 500 may be configured to detect the presence of by-products A stored at a location inside the storage tank 200 corresponding to the position of the sensor 500.

As illustrated in FIGS. 5 and 6, the sensor 500 may be configured to detect the presence of by-products A stored at a location inside the storage tank 200 corresponding to the position of the sensor 500 while moving between the first position and the central axis.

In addition, during the stirring operation by the stirring blade 400 which rotates together with the stirring rod 300, the by-products A fly and are deposited in the circumferential direction of the stirring rod 300 due to the centrifugal force caused by the rotation, and therefore the height of the by-products A may be different at a point near the central axis of the stirring rod and a point near the side wall spaced from the center.

Therefore, it is preferable that the guide rail 510 be formed vertically upwardly in the central axis direction of the stirring rod 300, as illustrated in FIG. 4A. This is because the vicinity of the central axis of the stirring rod is the most stable deposition location without the influence of centrifugal force, thereby improving the accuracy of deposition height measurement.

Accordingly, the sensor 500 is located on the center line of the stirring rod 300 on the outer side wall of the storage tank 200, thereby minimizing the influence of by-products A flying and depositing due to the stirring operation of the stirring blade 400, thereby providing the effect of improving the accuracy of deposition height measurement.

Such sensor 500 may be configured as a capacitive sensor, and the operation of the sensor 500 will be described below with reference to FIGS. 5 and 6.

As illustrated in FIG. 5, if the sensor 500 is sensing at the first position and the height of the by-products A stored in the storage tank 200 has not reached the first position, the change in electrostatic capacity of the sensor 500 will be small, and the controller will determine that the by-products A are not present at the first position.

However, as illustrated in FIG. 6, if the sensor 500 is sensing at the first position and the height of the by-products A stored in the storage tank 200 has reached the first position, the change in electrostatic capacity of the sensor 500 will be large, and the controller will determine that the by-products A are present at the first position.

In other words, the electrostatic capacity changes depending on the presence of the by-products A stored in the storage tank 200 at the position of the sensor 500, and by detecting the amount of change in electrostatic capacity, it is possible to detect that by-products A are present at a selected location.

The controller is connected to each component of the food waste disposer according to an embodiment of the present disclosure to control the food waste disposer, and may be configured to control the operation of the food waste disposer in conjunction with the sensor 500, and the display 600 may be configured to receive information from the controller and display the height of the by-products A or the state of the food waste disposer.

Specifically, the controller may be configured to determine that the by-products A are present up to the height of the first position in the case that the sensor 500 continuously detects the presence of the by-products A at a location inside the storage tank 200 corresponding to the position of the sensor 500 for a predetermined period of time at the first position where the sensor 500 is set.

In addition, when the controller determines that the by-products A are present up to the height of the first position, the controller may control the upper part of the housing 100 described above to be in a locked state, and the upper part of the housing 100 may be configured to be opened through unlocking by the user.

In addition, when the controller determines that the by-products A are present up to the height of the first position, the controller may be configured to display the height of the by-products A or that the storage tank of the food waste disposer is full on the display 600 described above.

In addition, the controller is provided with information on the deposition height or capacity inside the storage tank 200 according to the position of the sensor 500 in advance, and when the sensor 500 continuously detects the presence of the by-products A inside the storage tank 200 for a predetermined period of time, the controller may be configured to obtain the deposition height or capacity of the by-products A inside the storage tank 200 and display it on the display 600.

In addition, the controller may be configured to control the operation of the stirring rod 300 according to the opening and closing of the upper part of the housing 100, and specifically, it is preferable that the controller control the stirring rod 300 not to operate when the upper part of the housing 100 is open, and it is preferable that the stirring rod 300 is configured to operate when the upper part of the housing 100 is closed, but it is not necessarily limited thereto.

Further, it is preferable that the display 600 is configured to display a notification of the height of the by-products A inside the storage tank 200 in a visual or auditory manner, but it is not necessarily limited thereto.

The display 600 may be configured to display a notification using an LED, and specifically, when the sensor 500 detects by-products A stored in the storage tank 200, it is preferable to display a notification by changing the LED color depending on the position of the sensor 500, but is not necessarily limited thereto.

In addition, the display 600 may be configured to display a notification using a buzzer, and specifically, when the sensor 500 detects by-products A stored in the storage tank 200, it is preferable to display a notification by changing the buzzer sound or buzzer sound pattern depending on the position of the sensor 500, but is not necessarily limited thereto.

In addition, the display 600 may be configured to display a notification through a UI display screen, and specifically, when the sensor 500 detects by-products A stored in the storage tank 200, it is preferable to display at least one of the position of the sensor 500, the height of the by-products A stored in the storage tank 200, and the capacity, but is not necessarily limited thereto.

Accordingly, the food waste disposer with by-product emptying notification according to an embodiment of the present disclosure may detect by-products stored while food waste is fermented and decomposed inside a storage tank by a sensor, and display the height of the by-products or the state of the food waste disposer, thereby providing an effect of notifying the time to empty the by-products.

In addition, the food waste disposer with by-product emptying notification according to an embodiment may detect by-products stored inside a storage tank by a sensor located on an outer side wall of the storage tank rather than inside the storage tank, thereby providing an effect of improving the durability and accuracy of the detection sensor.

In addition, the food waste disposer with by-product emptying notification according to an embodiment may have a sensor located on a line passing through the central axis of a stirring rod on the outer side wall of the storage tank, thereby providing an effect of minimizing the influence of by-product deposition due to centrifugal force caused by the rotation of the stirring blade

In addition, the food waste disposer with by-product emptying notification according to an embodiment may have a sensor located on the central axis line of a stirring rod on the outer side wall of the storage tank, thereby minimizing the influence of by-products flying and depositing due to the stirring operation of the stirring blade, thereby providing an effect of improving the accuracy of deposition height measurement.

In addition, the food waste disposer with by-product emptying notification according to an embodiment has a sensor located on the outer side wall of the storage tank and configured such that its attachment position is adjustable, thereby providing the effect of sensing at various positions depending on the situation even with a single sensor.

Although the present disclosure has been described above with specific details such as specific components, limited embodiments, and drawings, these have been provided only to help a more general understanding of the present disclosure, and the present disclosure is not limited to the above embodiments, and those skilled in the art may attempt various modifications and variations from this description.

Therefore, the idea of the present disclosure should not be limited to the embodiments described above, and not only the appended claims but also all modifications equivalent to or equivalent to the claims are included in the scope of the idea of the present disclosure.

### EXPLANATION OF SYMBOLS

1000: Food waste disposer with by-product emptying notification according to an embodiment of the present disclosure
100: Housing
200: Storage tank
300: Stirring rod
400: Stirring blade
500: Sensor
510: Guide rail
600: Display
A: By-products of food waste

What is claimed is:

1. A food waste disposer with by-product emptying notification, the food waste disposer comprising:
a housing (100) having an upper part that opens and closes and a space formed inside;
a storage tank (200) coupled inside the housing (100) and configured to store by-products A of food waste;
a stirring rod (300) rotatably driven inside the storage tank (200);
at least one stirring blade (400) protrudingly formed on an outer circumferential surface of the stirring rod (300) and configured to stir the by-products A stored in the storage tank (200) by rotation of the stirring rod (300);
a sensor (500) configured to detect the by-products A inside the storage tank (200);
a controller configured to control an operation of the food waste disposer in conjunction with the sensor (500); and
a display (600) configured to receive information from the controller and display a height of the by-products A or a state of the food waste disposer,
wherein the sensor (500) is located on an outer side wall of the storage tank (200), and its attachment position is adjustable.

2. The food waste disposer according to claim 1, further comprising:
a guide rail (510) formed along an outer circumferential surface of the storage tank (200),
wherein the guide rail (510) is vertically formed in an upward direction of a central axis of the stirring rod (300), and
the sensor (500) is moved or fixed by sliding engagement with the guide rail (510).

3. The food waste disposer according to claim 1, wherein, at a first position where the sensor (500) is set,
in the case that the sensor (500) continuously detects the presence of the by-products A at a location inside the storage tank (200) corresponding to the position of the sensor (500) for a predetermined period of time,
the controller determines that the by-products A are present up to a height of the first position.

4. The food waste disposer according to claim 3, wherein, when the controller determines that the by-products A are present up to the height of the first position,
the controller controls the upper part of the housing (100) to be in a locked state, and
the upper part of the housing (100) is configured to be opened through unlocking by a user.

5. The food waste disposer according to claim 1, wherein the sensor (500) is configured to detect the presence of the by-products A stored at a location inside the storage tank (200) corresponding to a position of the sensor (500).

6. The food waste disposer according to claim 1, wherein the controller is provided with information on a deposition height or capacity inside the storage tank (200) according to a position of the sensor (500) in advance, and
in the case that the sensor (500) continuously detects the presence of the by-products A inside the storage tank (200) for a predetermined period of time,
the controller obtains the deposition height or capacity of the by-products A inside the storage tank (200) and displays it on the display (600).

7. The food waste disposer according to claim 1, wherein the sensor (500) is a capacitive sensor.

8. The food waste disposer according to claim 1, wherein the display (600) is configured to display a notification of the height of the by-products A inside the storage tank (200) in a visual or auditory manner.
